(19) United States
(12) Patent Application Publication
(10) Pub. No.: US 2014/0274975 A1
(43) Pub. Date: Sep. 18, 2014

(54) METHOD OF DETERMINING PRIOR HISTORY OF ISCHEMIC STROKE FOR CURRENT RISK EVALUATION AND THERAPY GUIDANCE

(71) Applicant: Health Diagnostic Laboratory, Inc., Richmond, VA (US)

(72) Inventors: Rebecca E. CAFFREY, North Chesterfield, VA (US); Mohmed ASHMAIG, Glen Allen, VA (US)

(73) Assignee: Health Diagnostic Laboratory, Inc., Richmond, VA (US)

(21) Appl. No.: 14/209,941

(22) Filed: Mar. 13, 2014

Related U.S. Application Data
(60) Provisional application No. 61/779,758, filed on Mar. 13, 2013.
(60) Provisional application No. 61/900,545, filed on Nov. 6, 2013.

(51) Int. Cl. G01N 33/543; A61K 31/519; A61K 31/4365; A61K 31/616

(52) U.S. Cl. CPC G01N 33/54306; A61K 31/616; A61K 31/519; A61K 31/4365
USPC 514/165; 436/501; 514/262.1; 514/301; 435/7.92

(57) ABSTRACT

The invention provides methods and compositions for diagnosing prior ischemic stroke or transient ischemic attack (TIA) and approximate the time said stroke occurred, comprising measurement of IgG and IgM antibodies to NR2A and/or NR2B NMDA receptor or fragment thereof in a biological sample, and comparing those measurements to reference population standards and to each other. The invention also includes optionally measuring other biomarkers for autoimmune disease for the reduction in false positives and better risk stratification for future stroke events. The method is particularly useful for identifying individuals that are at risk for future stroke or TIA, and for diagnosing previous history of ischemic stroke or TIA. This measurement and comparison enables determination of the risk of future stroke which is by definition higher in those patients who have suffered a previous stroke. The determining of existence of previous stroke and risk level of future stroke enable optimal therapeutic decisions to reduce risk of future events.

### CROSS-REFERENCE TO RELATED APPLICATIONS

This application claims the benefit of priority to U.S. Provisional Patent Application Ser. No. 61/779,758, filed Mar. 13, 2013 and U.S. Provisional Patent Application Ser. No. 61/900,545, filed Nov. 6, 2013, both of which are hereby incorporated by reference in their entirety.

### FIELD OF THE INVENTION

This application relates to methods of determining the existence of prior stroke and risk level of future stroke or transient ischemic attack (TIA). The methods also optionally include measuring at least one biomarkers for neurological disease, autoimmune disease, ischemic stroke and/or TIA and administering a preventive therapeutic regimen.

### BACKGROUND

Stroke is a leading cause of death and long-term disability in America and the western world. Despite many advances in diagnosis and treatment of acute events, accurate risk assessment and prevention remain the most effective approach to limiting damage to personal and public health. Subclinical and untreated ischemic events can be major factors for recurrence and/or indicators of poor outcomes in at-risk populations. See Pikula, A. et al., “Multiple biomarkers and risk of clinical and subclinical vascular brain injury: the Framingham Offspring Study,” Circulation, 125(17):2100-2107 (2012), which is hereby incorporated by reference in its entirety. According to Pikula, intensive methods such as MRI to detect subclinical brain injury reveal common prevalence of clinically undetected ischemic events in 28% of subjects over age 65 and 11% in Framingham study subjects under age 65. Even though the subjects do not suffer an acute clinical event, the undetected events are associated with cognitive decline, functional impairment, and importantly, substantially higher risk for subsequent stroke.

Transient ischemic attack (TIA) may be such a subclinical or undertreated pathophysiological event associated with detrimental outcomes in both the acute phase and in the longer term. Transient ischemic attack (TIA) and ischemic stroke are both caused by a clot, but in TIA, the blockage is temporary. Transient ischemic attacks (TIAs) are “warning strokes” that produce stroke-like symptoms but no lasting damage. TIAs are strong predictors of stroke. A person who has had one or more TIAs is almost 10 times more likely to have a stroke than someone of the same age and sex who has not. Recognizing and treating TIAs can reduce a patient's risk of a major stroke. See TIA (Transient Ischemic Attack), an updated article by the American Heart Association (Nov. 7, 2013), which is available at strokeassociation.org and is hereby incorporated by reference in its entirety.

A retrospective study by Lovett et al. (see Lovett, J. K. et al., “Very early risk of stroke after a first transient ischemic attack,” Stroke 34:e138-e140 (2003), which is hereby incorporated by reference in its entirety) illustrates the extent of the problem of untreated, undetected, or delays in treatment of TIA in the general population. Lovett et al. reports that more than 60% of patients with TIA take 3 days or more to see a physician, which is unsurprising given that only 8.6% of American adults could identify a symptom with TIA. It is estimated that about 5 million Americans have a diagnosis of TIA and that even more may have experienced an undiagnosed TIA. The risk of stroke is substantial in those having suffered a TIA, as described above, yet in a UK study, 54% of patients who experienced TIA did not receive medical attention until after their stroke. See Verro, P., “Editorial Comment-Stroke following TIA: mounting evidence of early risk,” Stroke 34:e141-e142 (2003), which is hereby incorporated by reference in its entirety.

In one study of those patients suffering a TIA, the risk of stroke within 2 days was 5.1%, 7 days was 10.3% and 30 days was 14.3%. See Lovett, J. K. et al., “Very early risk of stroke after a first transient ischemic attack,” Stroke, 34:e138-e140 (2003), which is hereby incorporated by reference in its entirety. There are few studies for such graded risks since, by their nature, the events and patients are undertreated for the initial event. An accurate assessment of prior ischemic events like TIA will both better assist researchers in understanding the problem and medical professionals in treating it.

If evidence of an ischemic episode exists, the elevated risk for a major event may require appropriate preventative care measures including lifestyle changes and clinical interventions can reduce the incidence of additional ischemic episodes, which is vital for subjects with a history of events.

Therapeutic methods and risk factors are well described in the art. Depending on the patient's risk, which can be informed by time since subclinical event, a medical professional may need to vary a patient's treatment from lifestyle recommendations to medication, hospitalization, or delay of a planned ancillary treatment such as endarterecotomy for those with cardiac stenosis. Therapeutic efforts may be substantially informed by a patient's other existing conditions or risk factors. Standard treatments to reduce the risk of future stroke include administration of anti-platelet agents such as aspirin. Patients with atrial fibrillation may be prescribed anticoagulants. Lifestyle changes are also necessary to reduce risk and associated conditions that increase risk may be treated to improve outcomes. The most important treatable factors linked to TIAs and stroke are high blood pressure, cigarette smoking, heart disease, carotid artery disease, diabetes, and heavy use of alcohol. A diagnosis of a subclinical event or TIA may be needed to justify or increase the urgency of such treatments. Effective and accurate laboratory blood tests for detection of TIA or stroke, or the risk of suffering a future TIA or stroke, could improve health outcomes for a substantial part of the population. See Svetlana A. Dambinova, U.S. Pat. No. 8,084,225, which is hereby incorporated by reference in its entirety.

A variety of markers of TIA and subclinical stroke have been evaluated for the diagnosis and risk assessment of future events. See e.g., Saenger, A. K. and Christenson, R. H., “Stroke biomarkers: progress and challenges for diagnosis, prognosis, differentiation, and treatment,” Clin. Chem. 56(1):21-33 (2010), which is hereby incorporated by reference in its entirety. Saenger describes the mechanisms and benefits of several markers including Lipoprotein-Associated Phospholipase 2 (Lp-PLA2), Asymmetric Dimethylarginine (ADMA), Matrix Metalloprotein 9 (MMP-9), S100 Beta, Glial fibrillary acidic protein (GFAP), and PARK7, which may have particular prognostic benefits for stroke risk and/or diagnostic relevance. Additional markers including those for inflammation such as C-reactive protein (CRP), VCAM-1, and MCP-1; hemostasis such as D-dimer, von Willebrand factor and plasminogen activator inhibitor-1; neurohormonal activity such as aldosterone-to-renin ratio, B-type brain natriuretic peptide (BNP), and N-terminal proatrial natriuretic peptide; endothelial function such as total homocysteine (tHcy) and urinary albumin/creatinine ratio (UACR); dyslipidemia and endothelial damage markers such as ApoC1, ApoC3, BNP, and FABP; growth factors such as BDNF; and endothelial damage markers such as MBP and NSE have also been studied for relevance in stroke diagnosis independently and as part of multi-marker panels. However, standard diagnostic methods commonly rely on physical and neurological examination and/or CT or MRI scans of the patient. See Pikula, A. et al., “Multiple biomarkers and risk of clinical and subclinical vascular brain injury: the Framingham Offspring Study,” Circulation 125(17):2100-2107 (2012); and Saenger, A. K. and Christenson, R. H., “Stroke biomarkers: progress and challenges for diagnosis, prognosis, differentiation, and treatment,” Clin. Chem. 56(1):21-33 (2010), both of which are hereby incorporated by reference in their entirety.

Multiple investigations have shown that N-methyl-D-aspartic acid (NMDA) receptors are overexpressed in the event of a stroke. See Dambinova, S.A. et al., “Multiple panel of biomarkers for TIA/stroke evaluation,” Stroke 33(5):1181-1182 (2002), which is hereby incorporated by reference in its entirety. NMDA receptors bind glutamate and typically contain 4 subunits, 2 NR1 and 2 NR2 subunits, and fragmentation of NR2 into NR2A and NR2B peptides is thought to occur with cerebral ischemia or neurotoxicity. Generation of NMDA receptor antibodies (NR2Abs) is mediated by the immune system following ischemic events, and either these autoantibodies or the NR2 peptide fragments themselves can be quantified in CSF and blood. Dambinova has shown in multiple studies that NR2Abs are detected in significantly higher quantities in the ischemic stroke and TIA patients vs controls with a high sensitivity and specificity.

Immunoglobulins M and G (IgM and IgG) are two classes of immunoglobulins whose properties can be used to evaluation timing of an antigenic challenge. IgM are antibodies produced immediately after an exposure to the antigen, and IgG are produced in a delayed response. For infectious disease, IgG generally confers immunity to a patient for a particular disease due to its continued presence long after the initial antigen exposure. In autoimmune disease, the body may produce antibodies against itself in error. IgM is the first antibody that is produced in case of an exposure to a particular antigen.

One difference between the two antibodies relates to exposure. While IgM is an indicator of a current or recent exposure to an antigen, an IgG indicates a recent or past exposure to the antigen. IgM is a temporary antibody that disappears within two or three weeks. It is then replaced by IgG which remains in the blood and provides lasting record of exposure to the antigen.

Autoimmune disorders generate autoantibody responses that may be accounted for in any diagnostic method measuring autoantibodies. Various autoimmune disorders may complicate NR2Ab measurement for TIA or stroke.

The detection of NMDA receptor autoantibodies for the diagnosis of autoimmune disorders is described in a variety of patents and applications. For example, PCT Publication No. WO/2012/076000, incorporated herein by reference in its entirety, describes the detection of IgA and/or IgM anti-NMDA receptor antibodies, and a method and an assay kit for the diagnosis of an autoimmune neuropathy, along with dementia, borderline personality disorder or primary psychosis.

U.S. Pat. No. 5,529,898, incorporated herein by reference in its entirety, also describes a method of screening a subject for a central nervous system disorder caused by autoimmune disease by detecting the presence or absence of NMDA receptor autoantibodies (called anti-glutamate receptor autoantibodies) in a biological sample. The presence of such autoantibodies indicates the subject is afflicted with a central nervous system disorder caused by autoimmune disease.

Patients with autoimmune diseases like Systemic Lupus Erythematosus (SLE) and Rheumatoid Arthritis (RA) are at higher risk for stroke by definition. This may partly be due to neurological and vascular components of these diseases in some individuals that may predispose them to stroke. Methods for diagnosing TIA or stroke can therefore account for autoimmunity factors that may otherwise generate an inaccurate analysis.

Tests exist to detect circulating NR2 subunits and autoantibodies generated to these subunits during the course of ischemic stroke including several patents by Dambinova. See, e.g., U.S. Pat. Nos. 6,896,872; 7,622,100; and 7,622,114; all of which are hereby incorporated by reference in their entirety.

U.S. Pat. No. 7,622,100 discloses a method for diagnosis of a stroke in a patient comprising directly or indirectly measuring within three hours after stroke onset the level of NR2A and/or NR2B NMDA receptor or fragment thereof in a subject. U.S. Pat. No. 7,622,114 discloses differentiation of ischemic from hemorrhagic stroke by measurement of NR2 subunits or autoantibodies to these subunits at the time the subject is suspected of suffering either type of stroke.

However, the tests and technologies derived from and disclosed by these patents generally involve detecting these analytes in an emergency room setting wherein (1) the patient samples are drawn within 3 hours of stroke onset; (2) agonists and antagonists of the analytes are also measured, and (3) the assays are performed using latex bead-based technology. The disadvantage to these techniques is that many patients with small strokes or TIAs do not seek emergency care, and thus samples may not be drawn until days or weeks after the event. Autoantibodies to NR2 or any autoantigen typically cannot develop within a 3 hour time period after a first stroke event. See “Immunology” 2nd edition, Roitt, Brostoff, and Male, 1989, Gower Medical Publishing, London, Chapter 8, which is hereby incorporated by reference in its entirety. It would usually take days for IgM-type autoantibodies to a primary challenge to be produced, and normally a week for IgG-type autoantibodies to be produced.

As noted in U.S. Pat. No. 6,896,872, “unfortunately, the use of NR2A and NR2B autoantibodies in the diagnosis of stroke or TIA does not provide a real-time assessment of the damage being done by a stroke or TIA. Rather, because of the time the immune system requires to mount an immune response, and to generate NR2A and NR2B autoantibodies, methods that test for these antibodies at best provide a delayed assessment of the extent and severity of stroke or TIA.” Dambinova does not take into account that the autoantibodies may have been generated from a previous event or previous underlying disease, such as an autoimmune disease. Also, the technique, as disclosed, does not provide for distinguishing subtypes of auto-antibody from each other (IgM vs. IgG) and comparing amounts thereof, but rather to global quantitation of all NR2 autoantibodies regardless of subtype. Thus, the techniques disclosed in Dambinova do not distinguish between strokes that happened days, weeks, or months ago, and do not distinguish 1st strokes from subsequent strokes. By not including at least one measurement of autoimmune disease markers in Dambinova, the presence of prior stroke from pre-existing autoimmune disease in either an acute care (emergency) setting, or a non-acute care, non-emergency setting wherein more than a day or two has passed since an ischemic stroke or TIA may not be easily be distinguished.

Beyond the critical inability to diagnose that a previous ischemic stroke or TIA has occurred in the past and determine the approximate amount of time in days and weeks that have passed since the event, the additional limitation of the stroke diagnosis technologies described above is that the methods do not lend themselves to multiplexing in a way that is faster, cheaper, more amendable to high-throughput methodologies for large-scale screenings of thousands of samples in a reference laboratory.

Therefore, there is a need in the art for an accurate immunological assay for the diagnosis of TIA and stroke that would also provide information on the risk of strokes in the future. This invention answers this need.

### SUMMARY OF THE INVENTION

The invention relates to a method of determining a subject's history of ischemic stroke or transient ischemic attack. The method includes the following steps: (a) measuring levels of NR2 subunit IgM autoantibodies and NR2 subunit IgG autoantibodies in a sample obtained from the subject; (b) comparing the measured levels of NR2 subunit IgM autoantibodies and NR2 subunit IgG autoantibodies from the sample to each other and/or to control levels of NR2 subunit IgM and IgG autoantibodies, respectively; and (c) determining a subject's history of ischemic stroke or transient ischemic attack based on the comparing step.

Examples of NR2 subunit autoantibodies may include NR2A subunit autoantibodies, NR2B subunit autoantibodies, and combinations thereof

The method further includes the step of detecting the presence or absence of at least one autoimmune disease biomarker in the sample, wherein the determining step is based on the detecting and comparing steps. The at least one autoimmune disease biomarkers may include an anti-nuclear antibody, a rheumatoid factor, an anti-Smith antibody, and an anti-RNP antibody.

The method also includes the step of detecting the presence or absence of at least one additional biomarker for ischemic stroke or transient ischemic attack in the sample. The method may further include the step of detecting the presence or absence of at least one biomarker of neurological disease in the sample. The determining step can be based on one or both of the above detecting and comparing steps.

The measuring step, as described above, may further include: (a) contacting the sample with one or more NR2 subunit antibody ligands coupled to a solid support under conditions effective for NR2 autoantibodies in the sample to bind to their NR2 subunit antibody ligand; (b) labeling bound NR2 autoantibodies with detectable anti-IgG or anti-IgM antibodies; and (c) detecting and distinguishing the labeled NR2 IgG and IgM autoantibody levels in the sample.

When the comparing step only shows that IgM NR2 subunit autoantibodies at a measurable level in the sample and IgG NR2 subunit antibodies are below a measurable level, it can be determined that the subject may have suffered a first ischemic stroke or transient ischemic attack less than 7-9 days prior to when the sample was obtained from the subject.

When the comparing step shows that the measured level of IgM NR2 subunit autoantibodies is higher than the measured level of IgG NR2 subunit autoantibodies in the sample, it can be determined that the subject may have suffered a first ischemic stroke or transient ischemic attack 7-10 days prior to when the sample was obtained from the subject.

If the comparing step shows that the measured level of IgG NR2 subunit autoantibodies is higher than the measured level of IgM NR2 subunit autoantibodies in the sample, it can be determined that the subject may have suffered a first ischemic stroke or transient ischemic attack between 10 days and 3 weeks prior to when the sample was obtained from the subject.

If the comparing step shows that the measured level of IgG NR2 subunit autoantibodies is greater than one log higher than the control level of IgG NR2 subunit autoantibodies, it can be determined that the subject may have suffered a secondary or tertiary ischemic stroke or transient ischemic attack.

The control levels of NR2 subunit IgM and IgG autoantibodies can be the average levels of NR2 subunit IgM and IgG autoantibodies in a clinical population and can also be NR2 subunit IgM and IgG autoantibody levels measured in the same subject at an earlier time point.

The subject can be asymptomatic for ischemic stroke or transient ischemic attack.

The method may further include assessing the subject's risk for future ischemic stroke or transient ischemic attack and/or administering a preventive therapeutic treatment regimen based on the determining step.

The invention also relates to a kit that includes (a) one or more NR2 subunit antibody ligands coupled to a solid support; (b) an anti-IgG antibody coupled to a first detectable label and (c) an anti-IgM antibody coupled to a second detectable label. NR2 subunit antibody ligands that can be included in the kit are NR2A subunit antibody ligands, NR2B subunit antibody ligands, and a combination thereof.

Additional aspects, advantages and features of the invention are set forth in this specification, and will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 is a rendering of a multiplex immunological assay for the detection of IgG and IgM targeted to NR2A and IgG and IgM targeted to NR2B.

FIG. 2 shows IgM and IgG responses to an antigenic challenge in titre vs. days.

FIG. 3 shows a diagnostic decision chart for evaluating the timing of an ischemic event in terms of IgM and IgG population.

### DETAILED DESCRIPTION OF THE INVENTION

It is an object of the invention to measure anti-NR2A/B autoantibodies, specifically IgM subtype, to detect first stroke or TIA in a patient who is not currently symptomatic, within days after the first event up to 2 weeks after the first event. Such measurement may allow for novel staging of historical stroke timeline.

It is also an object of the invention to use 2 anti-NR2A/B autoantibody subtypes (IgG and IgM) together in a panel with ratios described herein to inform historical timing of strokes. Previous tests have measured only aggregate antibody and cannot define a timeline.

It is also an object of the invention to use 2 anti-NR2A/B autoantibody subtypes (IgG and IgM) together in a panel with ratios described herein to distinguish 1st event from subsequent events in anamnestic response of IgG and IgM via relative magnitude of titer increase.

It is additionally an object of the invention to provide a multiplex assay of at least 4 measurements that include anti-NR2A IgG, anti-NR2A IgM, anti-NR2B IgG, and anti-NR2B IgM into a single assay, for reduced cost and reduced assay time/increased throughput.

It is an object of the invention to provide a biomarker panel with the addition of other biomarkers of ischemic stroke as well as other biomarkers for disease conditions that may also generate anti-NR2 auto-antibody, such as SLE and RA and some forms of encephalitis. These additional biomarkers may confer added specificity to the measurement of anti-NR2 autoantibodies, by identifying which patients may be “false positives” for stroke. Addition of at least one biomarker for autoimmune disease may confer more information on increased risk for stroke/TIA. The addition of at least 1 additional autoimmune disease and/or other biomarkers to enable appropriate diagnosis and treatment of underlying conditions that may predispose a patient to increased risk of stroke; treatment of underlying condition in addition to standard preventative therapies may further lower patient risk of stroke.

The addition of other biomarkers of ischemic stroke, as well as other biomarkers for disease conditions that may also generate anti-NR2 auto-antibody, such as SLE, RA and some forms of encephalitis would provide more accuracy in the diagnostic or prognostic assay. Inclusion of tests including but not limited to Anti-Nuclear Antibody (ANA), Rheumatoid Factor (RF), anti-Smith (Anti-Sm) antibody, or similar additional biomarkers may confer added specificity to the measurement of anti-NR2 autoantibodies, by identifying which patients may be present as false positive for stroke. These determinations, if positive, could mean that in an asymptomatic patient, an ischemic stroke may be more likely to occur than in a patient who is negative for these determinations. It may also indicate a false negative for prior stroke. These determinations would point out the need to treat an underlying auto-immune condition to ameliorate the risk that such condition could precipitate a future cardiovascular event such as ischemic stroke. The addition of at least 1 additional biomarker would enable appropriate diagnosis and treatment of underlying conditions that may predispose a patient to increased risk of stroke; treatment of underlying condition in addition to standard preventative therapies further lowers patient risk of stroke.

Additional markers can avoid the generation of a false negative result because of the unique properties of various autoimmune disorders. For example, many patients who have rheumatoid disease are positive for Rheumatoid Factor (RF). RF is essentially human anti-human antibody, in other words, a person makes antibodies that bind to the Fc Portion of human IgG antibodies, which can interfere with a diagnostic assay. Patients who are RF+ often test as false positives on many different types of assays because detection antibodies often bind to Fc portions of capture antibodies. Detection of RF in a panel would avoid a false negative result for NR2 Abs.

An additional complicating factor is the association of multiple autoimmune diseases that present multiple antibodies to complicate analysis. For example, many people who are RF+ also have Lupus, and therefore may also be positive for anti-NR2 antibodies (true positive) as a result of their disease disrupting blood-brain barrier or because they have antibodies that cross-react with NR-2. Results of testing in patients with autoimmune disease are usually interpreted with great caution, repeated often, and confirmed by other means, if possible. Additional markers in a diagnostic panel of NR2Ab are valuable in making a correct diagnostic assessment.

In one embodiment shown in FIG. 1, technology such as the Luminex bead-based multiplex assay will be used to multiplex simultaneous detection of both IgG and IgM subtypes of anti-NR2A and anti-NR2B autoantibodies. In a single determination, 2 differently colored beads carrying NR2A and NR2B protein or peptide, respectively, are mixed with a biological sample obtained from a patient who is not experiencing acute symptoms of a stroke or TIA at the time the sample is drawn. The biological sample is then incubated such that sufficient time passes for autoantibodies to the NR2 antigens to bind to the antigens on the beads. The beads are washed, and differentially labeled anti-human IgG or IgM is added and allowed to bind to the previously bound autoantibody(s) already captured on the beads. The labels are detected. The relative amounts of IgG and IgM bound to the NR2A and NR2B beads are measured, and the amount measured is compared to a population reference standard, in addition to the ratio of IgG to IgM being calculated for each NR2 subtype. The presence, absolute amounts compared to standards, and relative amounts of the 2 subtypes of autoantibody are then used to determine 1) whether a previous stroke has occurred, and 2) about how long ago in days, weeks, or months the stroke occurred, and 3) whether there have been multiple stroke events. This information can be used for patient management therapy decisions to treat appropriately and reduce risk of future stroke.

In a more general qualitative assay, semi-quantitative ELISAs with commercially-available monoclonal antibodies as calibrators are constructed for detection of NR2Abs. 2 individual semi-quantitative ELISAs are used: one comprising NR2A and NR2B antigen for IgG capture and one comprising the NR2A and NR2B antigen for IgM capture. Intact NMDA, NR2 peptide, and/or recombinant NR2A and NR2B is bound to beads for the capture of anti-NR2 IgG and IgM, respectively. Goat or rabbit anti-human IgG Fc and anti-human IgM Fc with fluorescent HRP label can be used to detect the bound IgG or IgM. Results may indicate the presence or absence of IgG and/or IgM to the NR2 sequence of interest and relative intensity gives an indication of the stage of the disease.

In a fully quantitative assay, an accurate calibrator is required for accurate assessments. A monoclonal chimeric antibody is included (mouse immunization, harvesting of spleen cells, fusion with myeloma cell line, and culture to isolate clones). All potential clones (high titer) are screened by ELISA and subcloned by limiting dilution. A Master Cell Line will be cryopreserved. A clone is then purified and chimerized to provide a human Fc region. Intact NMDA, NR2 peptide, and/or recombinant NR2A and NR2B may be used for capture of the patient antibodies. Goat or rabbit anti-human IgG Fc and anti-human IgM Fc with fluorescent HRP label or with different fluorescent labels can be used in the multiplex assay for detecting bound antibodies. Chimeric mouse monoclonal antibody to NR2A and NR2B (both IgG and IgM) with human Fc region are needed as the first and second calibrators. A patient's serum sample is passed over the NR2 beads, washed, and detected with the fluorescently-labeled antibodies. Known concentrations are simultaneously run with the calibrator antibodies for comparison and calculation of the patient's IgG and IgM levels.

FIG. 2 shows generalized antibody responses to an antigenic challenge over time, adapted from “Immunology” 2nd edition, Roitt, Brostoff, and Male, 1989, Gower Medical Publishing, London, Chapter 8, Cell Cooperation in the Antibody Response, page 8.1, FIGS. 8.1 and 8.2, which is hereby incorporated by reference in its entirety. It is known in the art that when an antigen is encountered by the human immune system, IgM is the first antibody subtype to be produced. Depending on the antigen and the dose, IgM will be produced within days, and appears before IgG. IgG antibody typically appears at detectable levels after IgM, for most antigens at around 7 days. The IgM antibody response peaks typically just after 1 week of antigen administration and then begins to decline as the IgG titer to the antigen rises. The IgG titer to a primary immune challenge tends to peak around 2 weeks after challenge, then decline slowly and remain at detectable levels. IgM may often decline to very low levels, at least one log titer lower than IgG, around this 2-week time point.

If a secondary antigen challenge occurs, in this case a 2nd stroke or TIA, the secondary (anamnestic) response occurs, and this is a much more robust response with much higher antibody titers, particularly of IgG, that may reach up to 4-5 logs higher titers than reference population controls and typically can be ten-fold and sometimes up to 2-3 logs higher than the antibody titers measured in a primary response. Additionally, IgM may increase to detectable levels during a secondary response. Thus, by measuring the presence/absence and relative expression of anti-NR2 auto-antibodies, it may be possible to determine whether and when a previous stroke or TIA occurred, assess risk of future events, and treat accordingly.

As an example, if IgM is present and IgG absent, the ischemic stroke occurred no longer than 7-9 days prior to the sample being drawn. If IgG is also present, but the IgM titer is higher, then the stroke is a first stroke, and may have occurred 7-10 days previously. If the IgG and IgM are both present, but IgG is higher, and within one log of the antibody titer of a negative reference population, it has been longer than 10 days and probably less than 2 or 3 weeks since the first occurrence of stroke. If IgG is detectable at a level that is less than one log above the reference range, and IgM is undetectable or near the LOD, probably at least 2 or at least 3 weeks may have passed since the first stroke. If IgG is present at least 1 log above reference range or previous primary immune response, and particularly if IgG titer is at least 1 log higher than IgM titer (if IgM is present), then it is likely that a secondary or tertiary event may have occurred.

The case in which IgG is present at least 1 log over reference, prior reading or IgM can be at any time point since the initial ischemic event, because in a secondary immune challenge (as in a second or third stroke) one may have an anamnestic immune response. Since the subject may have already went through an affinity maturation process and there may now have circulating memory B cells that produce high-affinity IgG anti-NR2 antibodies from a prior stroke event (first inoculation with NR2 antigen), when the subject is re-challenged with the antigen again he or she may very quickly produce copious quantities of high-affinity IgG.

In an additional embodiment, if comparing IgG and IgM levels to each other or to standards shows that the measured level of IgG NR2 subunit autoantibodies is greater than one log higher than the control level of IgG NR2 subunit autoantibodies, it is determined that the subject may have had an anamnestic response to a repeat endogenous inoculation of NR-2 antigen in the course of a secondary or tertiary ischemic stroke or transient ischemic attack. In that case, the patient may require more aggressive treatment even without presenting an acute condition of stroke or TIA.

In various embodiments, the method can be used in a clinical setting to determine an individual's risk of stroke, to select appropriate therapies, and/or to monitor the effectiveness of risk reduction therapies. In one embodiment, the baseline levels may be derived from population averages. In another embodiment, the baseline levels may be derived from the individual's own medical history. In another embodiment, the method can be performed repeatedly (i.e. more than once) to monitor the reduction or increase in risk for stroke or TIA, optionally in conjunction with the administration of any risk reduction therapy.

FIG. 3 includes one embodiment of the invention which involves a diagnostic chart 100 for the evaluation of a patient's time since ischemic event by anti-NR2 IgG and IgM levels. In 101, a patient sample is analyzed according to the ELISA assay for anti-NR2, or anti-NR2A and anti-NR2B IgG and IgM. In 102, the IgG and IgM outputs are recovered and compared to each other. In case 1, 103, IgM NR2 subunit autoantibodies are at a measurable level in the sample and IgG NR2 subunit antibodies are below a measurable level, leading to result 108, where it is determined that the subject suffered a first ischemic stroke or transient ischemic attack less than 7-9 days prior to when the sample was obtained from the subject. In case 2, 104, the measured level of IgM NR2 subunit autoantibodies is higher than the measured level of IgG NR2 subunit autoantibodies in the sample, leading to result 109, where it is determined that the subject suffered a first ischemic stroke or transient ischemic attack 7-10 days prior to when the sample was obtained from the subject. In case 3, 105 the measured level of IgG NR2 subunit autoantibodies is higher than the measured level of IgM NR2 subunit autoantibodies in the sample. This situation may involve a difference smaller than a log titre, 106, leading to result 110, where it is determined that the subject suffered a first ischemic stroke or transient ischemic attack between 10 days and 3 weeks prior to when the sample was obtained from the subject or it may involve IgG NR2 subunit autoantibodies greater than one log higher than the control level of IgG NR2 subunit autoantibodies, leading to result 111, where it is determined that the subject suffered a secondary or tertiary ischemic stroke or transient ischemic attack. All results 108-111 may indicate different levels of urgency and comparison to marker values for autoimmune disorders or associated cardiometabolic conditions 112, informing a treatment protocol administered by a medical professional in 113.

In an additional embodiment, measurements of anti-NR2A/B autoantibodies of both subtypes could be carried out by binding their respective antigens to any solid support known in the art, or by nephelometry or liquid supports such as capillary electrophoresis (CE), or by measurements of fluorescence quenching similar to Förster resonance energy transfer (FRET). In one embodiment, Luminex multiplexing is used for measuring anti-NR2A/B autoantibodies of both subtypes and fluorescently tagged antibodies are used as the method of detection. One Luminex-type method would be based on magnetic beads, which are usually silica or dextran beads with antigen bound to the surface; this embodiment may be advantageous over, for example, latex beads, because latex beads can be lost during washing steps, reducing the signal and making detection of small amounts of analyte more difficult. Magnetic beads are retained in place by a magnet such that unbound material is efficiently washed away in wash steps, without loss of the beads. Thus, the signal is stronger, and there is less “noise” from incompletely washed away labeled secondary antibody.

In other embodiments, the measurement of auto-antibody can be made by binding antigen to an ELISA plate or to beads that are not colored and magnetic, and separate determinations (not multiplexed) could be made of IgG and IgM, rather than all at once. In one embodiment, the secondary labeled antibodies may be labeled with a non-fluorescent compound such as HRP, biotin, or similar labels and the detection event would occur by colorimetric chemical reaction or binding to a tertiary (i.e., labeled avidin or streptavidin). In yet another embodiment, the assay method may be automated and steps from the addition of sample and washing can be automated on robotics; further, the labeling and detection may be automated, and the data analysis and reporting steps may be automated for maximum throughput (thousands of samples per day) and minimal time per assay. The calculation of IgG/IgM ratios and all individual measurements may be flagged as optimal/low risk, intermediate risk, or high risk by software and reported thusly in an electronic or paper format to a healthcare provider or patient.

In yet another embodiment, other biomarkers can also be measured to assess the risk for stroke or TIA, prognose, select therapies, and monitor therapeutic progress. In some variations of the embodiments, other biomarkers may be further multiplexed, such as the addition of colored magnetic beads specific for the capture of at least one, at least “n−1”, and at least “n”, of a list of biomarkers for autoimmune diseases, other neurological conditions, and other biomarkers for ischemic stroke known to those in the art, comprised of “n” members, wherein the diseases and conditions (such as SLE, RA and types of encephalitis) are known to generate anti-NR2 auto-antibodies in some patients; the determined values of these additional biomarkers would be correlated with risk of future ischemic stroke to better inform the predictive/prognostic value of the invention. In other embodiments, the biomarkers from the list comprised of “n” biomarkers would be measured separately (not multiplexed), but their determined values would be also correlated to risk of future ischemic stroke event for the reason already stated.

The term “subject” as used herein includes, without limitation, mammals, such as humans or non-human animals. Non-human animals may include non-human primates, farm animals, sports animals, rodents or pets. A typical subject is human and may be referred to as a patient. Mammals other than humans can be advantageously used as subjects that represent animal models of the cardiovascular disease or for veterinarian applications.

A “biological sample” encompasses a variety of sample types obtained from a subject with a biological origin. Examples of biological fluid sample include, but are not limited to, blood, cerebral spinal fluid (CSF), interstitial fluid, urine, sputum, saliva, mucous, stool, lymphatic, or any other secretion, excretion, or and other bodily liquid samples. Exemplary biological fluid sample can be a blood component such as plasma, serum, red blood cells, whole blood, platelets, white blood cells, or components or mixtures thereof.

There are many therapeutic alternatives that can be employed to reduce the risk of stroke in an individual. Anti-platelet agents such as aspirin or Plavix are routinely prescribed for patients at risk for stroke. Patients with AFIB (atrial fibrillation) are at greater risk of stroke than the general population and also may be prescribed various anticoagulants, or treated with surgical procedures. Autoimmune diseases and other neurological diseases may also predispose patients to higher risk of stroke/TIA. Therefore, in some embodiments, the invention can be used to diagnose and treat an autoimmune condition or a neurological condition by administering therapies known and practiced by those familiar with the art, for the purpose of lowering risk of stroke/TIA. In this embodiment, monitoring of the patient's comorbid disease condition together with the anti-NR2A/B autoantibodies can further inform risk analysis and treatment guidance.

In other embodiments, based upon results showing an increased risk of suffering TIA or stroke, preventative therapy can be administered. In another embodiment, the tests described herein can be used to monitor the patient's condition with respect to changes in risk level, or to monitor the effectiveness of a given therapy using the methods of the present invention. Preventive therapy may involve treating comorbidities such as high blood pressure, quitting smoking, improving blood lipids and inflammatory conditions, insulin resistance/diabetes, and prescribing lifestyle changes such as diet, weight loss, and exercise programs all lower risk of future stroke/TIA. The biomarker tests, as described herein, can be used to monitor response to these and other therapies.

In some cases, preventative therapy may include aspirin alone, dual anti-platelet therapy with aspirin and dipyridamole or clopidogrel, which are known to reduce stroke risk and recurrent events. Extended-release dipyridamole with aspirin may be useful soon after or long after a stroke or TIA. Dipyridamole or clopidogrel may be added to an initial administration of aspirin. Alternatively, aspirin may be avoided if a patient is also at higher risk of bleeding.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

All publications and patent applications mentioned in this specification are herein incorporated by reference in their entirety to the same extent as if each individual publication or patent application was specifically and individually indicated to be incorporated by reference.

What is claimed is:

1. A method of determining a subject's history of ischemic stroke or transient ischemic attack, said method comprising:
measuring levels of NR2 subunit IgM autoantibodies and NR2 subunit IgG autoantibodies in a sample obtained from the subject;
comparing the measured levels of NR2 subunit IgM autoantibodies and NR2 subunit IgG autoantibodies from the sample to each other and/or to control levels of NR2 subunit IgM and IgG autoantibodies, respectively; and
determining a subject's history of ischemic stroke or transient ischemic attack based on said comparing.

2. The method of claim 1, wherein the NR2 subunit autoantibodies are selected from NR2A subunit autoantibodies, NR2B subunit autoantibodies, and combinations thereof.

3. The method of claim 1 further comprising:
detecting the presence or absence of at least one autoimmune disease biomarker in the sample, wherein said determining is based on said detecting and said comparing.

4. The method of claim 3, wherein the at least one autoimmune disease biomarker is selected from the group consisting of an anti-nuclear antibody, rheumatoid factor, an anti-Smith antibody, and an anti-RNP antibody.

5. The method of claim 1 further comprising:
detecting the presence or absence of at least one additional biomarker for ischemic stroke or transient ischemic attack in the sample, wherein said determining is based on said detecting and said comparing.

6. The method of claim 1 further comprising:
detecting the presence or absence of at least one biomarker of neurological disease in the sample, wherein said determining is based on said detecting and said comparing.

7. The method of claim 1, wherein said measuring comprises:
contacting the sample with one or more NR2 subunit antibody ligands coupled to a solid support under conditions effective for NR2 autoantibodies in the sample to bind to their NR2 subunit antibody ligand;
labeling bound NR2 autoantibodies with detectable anti-IgG or anti-IgM antibodies; and
detecting and distinguishing the labeled NR2 IgG and IgM autoantibody levels in the sample.

8. The method of claim 1, wherein the comparing step shows only IgM NR2 subunit autoantibodies are at a measurable level in the sample and IgG NR2 subunit antibodies are below a measurable level, whereby it is determined that the subject suffered a first ischemic stroke or transient ischemic attack less than 7-9 days prior to when the sample was obtained from the subject.

9. The method of claim 1, wherein the comparing step shows that the measured level of IgM NR2 subunit autoantibodies is higher than the measured level of IgG NR2 subunit autoantibodies in the sample, whereby it is determined that the subject suffered a first ischemic stroke or transient ischemic attack 7-10 days prior to when the sample was obtained from the subject.

10. The method of claim 1, wherein the comparing step shows that the measured level of IgG NR2 subunit autoantibodies is higher than the measured level of IgM NR2 subunit autoantibodies in the sample, whereby is determined that the subject suffered a first ischemic stroke or transient ischemic attack between 10 days and 3 weeks prior to when the sample was obtained from the subject.

11. The method of claim 1, wherein the comparing step shows that the measured level of IgG NR2 subunit autoantibodies is greater than one log higher than the control level of IgG NR2 subunit autoantibodies, whereby it is determined that the subject suffered a secondary or tertiary ischemic stroke or transient ischemic attack.

12. The method of claim 1, wherein the control levels of NR2 subunit IgM and IgG autoantibodies are average levels of NR2 subunit IgM and IgG autoantibodies in a clinical population.

13. The method of claim 1, wherein the control levels of NR2 subunit IgM and IgG autoantibodies are NR2 subunit IgM and IgG autoantibody levels measured in the same subject at an earlier time point.

14. The method of claim 1, wherein said subject is asymptomatic for ischemic stroke or transient ischemic attack.

15. The method of claim 1 further comprising:
assessing the subject's risk for future ischemic stroke or transient ischemic attack based on said determining.

16. The method of claim 1 further comprising:
administering a preventive therapeutic treatment regimen based on said determining.

17. A kit comprising:
one or more NR2 subunit antibody ligands coupled to a solid support an anti-IgG antibody coupled to a first detectable label and an anti-IgM antibody coupled to a second detectable label.

18. The kit of claim 17, wherein the NR2 subunit antibody ligands are selected from NR2A subunit antibody ligands, NR2B subunit antibody ligands, and a combination thereof.
